(19) United States
(12) Patent Application Publication
(10) Pub. No.: US 2023/0380763 A1
(43) Pub. Date: Nov. 30, 2023

(54) PHYSICAL THERAPY MONITORING SYSTEM

(71) Applicant: TAU Orthopedics, Inc., Rancho Santa Fe, CA (US)

(72) Inventors: Anthony DeSena, Rancho Santa Fe, CA (US); Gerard von Hoffmann, Rancho Santa Fe, CA (US)

(21) Appl. No.: 18/311,034

(22) Filed: May 2, 2023

Related U.S. Application Data
(60) Provisional application No. 63/364,261, filed on May 5, 2022.

(51) Int. Cl. A61B 5/00; A61B 5/11

(52) U.S. Cl. CPC A61B 5/6812; A61B 5/112; A61B 5/4571; A61B 5/4585; A61B 5/4595; A61B 2505/09; A61B 2560/0443

(57) ABSTRACT

Disclosed is a wearable physical therapy data collection system. The system includes a wearable support having a waistband and at least one motion detector assembly. The motion detector assembly includes a femoral strut extending between a superior end and an inferior end; a polyaxial connection between the superior end and the waistband; and hip sensors in motion sensing communication with the polyaxial connection, configured to capture data reflecting movement of the femur with respect to the hip in the medial—lateral direction, anterior—posterior direction and rotation of the femur. A tibial strut may extend between the inferior end and a polyaxial connection to a foot attachment. Sensors may be provided to capture data reflecting tibial rotation, and flexion, extension, rotation and pronation of the foot. Separate left and right motion detectors enable collection of bilateral data, to identify bilateral imbalances and monitor rehabilitation progress.

### BACKGROUND OF THE INVENTION

Many acute, chronic, and congenital disease states have a significant impact on the musculoskeletal system in either a direct way, e.g., primary musculoskeletal disorders; or, in an indirect way as a manifestation of musculoskeletal injury, deconditioning, atrophy, or disability. From the perspective of diagnosis, a key component of the medical evaluation may include a focused evaluation of musculoskeletal performance. These include but are not limited to neurologic disorders, bone and joint disease, and injury/trauma. From the therapeutic perspective, specific, therapeutic grade musculoskeletal maneuvers are key components of treatment for musculoskeletal, neurologic, cardiovascular, and pulmonary disease states. Usually grouped under the category of “rehabilitation” therapy, or rehab for shorthand terminology, which are used interchangeably herein, these maneuvers can employ techniques used by, but not limited to, physical therapists, occupational therapists, and speech pathologists to improve the overall efficacy of medical and surgical therapy by counteracting physical deconditioning, building/maintaining musculoskeletal strength, range of motion, coordination/balance, and facilitating pain relief. Example patient populations that benefit from rehab include those with primary musculoskeletal problems such as osteoarthritis, rheumatoid arthritis, disability from stroke, and patients who have undergone musculoskeletal surgery as well as patients with obesity, cardiovascular disease, and pulmonary diseases such as myocardial infarction and chronic obstructive pulmonary disease.

Rehab therapy is prescribed to patients by medical professionals and depends upon various healthcare professionals including physical therapists, occupational therapists, and physicians for care delivery. However, there are significant challenges to current models of rehab delivery and implementation. Among them are, first, traditional rehab may be cost prohibitive for both short term and long term (maintenance) use. Second, logistical difficulties may prevent patients from engaging the current outpatient rehab clinical network in person, due to pandemic shutdowns or other logistics. Third, rehab exercises may be difficult to understand and perform correctly without coaching. Third, preventative therapy and exercise is not something covered by insurances, despite its proven ability to prevent injury and deconditioning.

Established protocols in the form of specific musculoskeletal exercises are currently administered to patients in a variety of both outpatient and inpatient settings. These are typically initially administered via trained medical professionals (physical therapists, occupational therapists, rheumatologists, internal medicine physicians, orthopedic surgeons) during designated sessions, typically four to six weeks, with sessions typically lasting 45-60 minutes. At intermittent points throughout the rehab therapy progress, both ‘objective’ (e.g., goniometer estimation of range of motion) and subjective measures are obtained and checked by clinic visits or visits by the relevant rehab professionals.

Outside of supervised, outpatient rehab centers, patients are encouraged to perform the appropriate rehab maneuvers at home, typically using passive instructional paper handouts that display a sequence of printed exercises. Additionally, patients may have access to digital rehab content delivered through digital media and/or downloaded from internet rehab providers that consist of animations and/or videos of rehab maneuvers.

Despite these proposed solutions, a number of problems remain, resulting in less-than-optimal rehabilitation participation and outcomes including slow recovery and longer time out of the workforce among other consequences. Unsupervised rehabilitation with only qualitative feedback on progress may become burdensome and unengaging causing treatment adherence to decline over time. In office based rehab can be cost prohibitive. Self reporting of compliance with assigned protocols is notoriously unreliable, impairing the physician's ability to optimize the patient's therapy.

Home exercise sessions carry issues of proper execution and the provision of useful feedback. Unless rehab exercises are supervised by trained clinicians, patients do not receive proper and timely feedback while executing specific maneuvers. Thus, patients may adopt improper technique for rehab exercises thereby reducing the therapeutic benefit of the exercise and possibly increasing the risk of injury. Even in an office visit, tracking rehab progress is largely qualitative and can be difficult. The lack of routine, quantitative measurements of patients' motion during rehab exercises, especially in the home rehab setting, makes tracking patient progress in serial rehab difficult. This in turn makes it difficult to assess the efficacy of the prescribed rehab regimen.

Early and safe ambulation is important to patient recovery. If done correctly, it can abbreviate the duration of a patient's hospital stay and produce meaningful economic benefits as well as more rapidly returning the patient to their normal life. Methods to encourage and measure patient ambulation are few, which is why there remains a need for ambulatory patient gait tracking capabilities. Workplace applications such as minimizing the risk of injury or work hardening and FCE can also produce meaningful savings to the various stakeholders involved.

Notwithstanding prior advances, there remains a need for improvements in the rehabilitation and patient monitoring processes, in both the clinic and home based environments.

### SUMMARY OF THE INVENTION

There is provided in accordance with one aspect of the invention, a wearable rehabilitation system. The system comprises a wearable support comprising a waistband and at least one motion detector. The motion detector comprises a femoral strut extending between a superior end and an inferior end; a polyaxial connection between the superior end and the waistband; and hip sensors in motion sensing communication with the polyaxial connection, configured to capture data reflecting movement of the femur with respect to the hip in the medial—lateral direction, anterior—posterior direction and rotation of the femur.

The wearable rehabilitation system may comprise a left leg motion detector and a right leg motion detector. The system may further comprise a knee attachment configured to secure the inferior end of the femoral strut with respect to the knee. The knee attachment may comprise a flexible cuff. The system may further comprise a tibial strut extending between the knee attachment and an inferior attachment configured to secure the tibial strut with respect to the ankle. The inferior attachment may be a flexible cuff for surrounding the ankle and/or foot.

The system may additionally comprise a foot attachment, for securing the tibial strut with respect to a wearer's foot. At least one sensor may be provided for measuring pronation and supination of the foot. At least one sensor may be provided for measuring dorsiflexion and plantarflexion of the foot.

The length of the femoral strut between the waistband and the knee attachment may be adjustable, and the femoral strut may comprise first and second telescopically extendable components. The first and second components may be tubular to provide a protected pathway for wiring.

The wearable physical therapy system may additionally comprise a knee module carried by the knee attachment. At least one sensor may be provided in the knee module, configured to measure flexion and extension at the knee. Electrical conductors may extend through the femoral strut to connect the knee module to a hip module.

Sensors at one or more of a hip module, knee module and ankle module may collect angular position and time data. The sensor may comprise a rotary encoder. At least one of a hip module and a knee module includes a sensor configured to determine femoral rotation, and at least one sensor is configured to determine range of motion.

In accordance with another aspect of the invention, there is provided a wearable ankle range of motion measuring device. The device comprises a foot support, configured for attachment to a foot, and a tibial support, configured for attachment to the lower leg. A polyaxial connection is provided between the tibial support and the foot support, having a first sensor for measuring medial rotation and lateral rotation, a second sensor for measuring dorsiflexion and plantar flexion, and a third sensor for measuring inversion and eversion.

At least one sensor comprises an angular position and time sensor, and may be a rotary encoder. The foot support may comprise a foot plate configured for attachment to a top or a bottom of a foot. The tibial support may be attached to the leg at the knee. The wearable ankle range of motion measuring device may be provided in combination with a tibial rotation sensor. The wearable ankle range of motion measuring device may further comprise a waistband and a femoral strut extending between the waistband and the knee. A hip module may be provided with at least one sensor for measuring range of motion at the hip, and a knee module may be provided with at least one sensor for measuring range of motion at the knee.

There is also disclosed a computer-implemented method of displaying physical therapy progress to a patient and/or therapist. The method comprises, via at least one processor of at least one computing platform operably coupled to at least one storage device and a display device: providing the at least one storage device containing a first set of user body movements of a user performing a movement routine previously captured via at least one motion tracking apparatus; capturing a second set of user body movements of the user performing the movement routine; and presenting data to the user via the display device, the data configured to display a difference between the first and second sets of user body movements to reveal physical therapy progress on metrics such as range of motion, femoral or tibial rotation, or any of the others disclosed herein. Progress feedback can also be provided through an audible interface for patients who are vision impaired.

The computer-implemented method may additionally comprise storing user body movements captured via the at least one motion tracking apparatus in the at least one storage device as benchmark information for the user. The method may include displaying data via at least one wireless link to a remote therapist.

There is also provided a computer-implemented method, comprising, via at least one processor of at least one computing platform operably coupled to a display device: presenting an avatar on the display device configured to perform instructional body movements indicating instructions for performing a movement routine. Body movement data is captured from a patient seeking to replicate motion of the avatar. An indication of a degree of compliance between the body movement data and motion of the avatar may be presented via a wired or wireless connection to a local display in the case of an office based therapy session, or via remote display in the case of a telemedicine therapy session. Progress feedback can also be provided through an audible interface for patients who are vision impaired.

There is also provided a wearable feedback support for coaching a wearer to replicate a predetermined body motion pattern, comprising: a wearable support; a plurality of sensors carried by the support; a plurality of effectors carried by the support for providing feedback to a wearer; a controller, including a processor and a memory configured to store body motion reference data; wherein the processor is configured to activate an effector to provide vibrational feedback to the wearer whether a body position achieved by the wearer has achieved alignment with a body position reference data. The reference data may reflect a previously achieved body position, enabling the quantification of progress, or a body position goal input by the patient or by a health care provider. The reference data may comprise a target range of motion.

The feedback support may include an effector on one or more of the anterior right side of the support, on the anterior left side of the support, on the posterior right side of the support, and or on the posterior left side of the garment.

The feedback may be configured to train the wearer to follow a predetermined routine. The controller may further comprise communication electronics for wireless communication with a remote device. The remote device may comprise a smart phone, or a remote work statin such as a display accessible by a remote health care provider.

The reference data may comprise the wearer's previously stored data, or may comprise data associated with a performance objective.

The controller may be configured to transmit feedback to the wearer substantially in real time as the wearer's body motion achieves the performance objective. The reference data may relate to a wearer's range of motion, and the feedback may include a visual representation of the wearer's body.

The system of the present invention enables the physical therapy session to be moved from the clinical environment to the patient's home or other remote location. In one implementation, the patient and therapist can be communicatively connected in real time such as by telephone or by way of a communication channel integrated into a system UI/UX. The therapist can instruct the patient to perform certain motions and the range of motion or other data can be observed by the therapist in real time, in large part reproducing the in clinic experience.

Optionally the patient can film themselves such as on a smartphone, and transmit video to the therapist so the therapist can observe the motion on a monitor and the corresponding data in real time. Alternatively, the patient can wear the system and undertake a predetermined movement protocol assigned by the therapist. Data can be recorded and transmitted to the therapist for review at a later time.

### BRIEF DESCRIPTION OF THE DRAWINGS

FIG. 1A is a front elevational schematic view of a physical therapy monitoring system in accordance with the present invention.

FIG. 1B is a side elevational view of the system of FIG. 1.

FIG. 1C Is a front elevational illustration of one implementation of the invention configured for bilateral data capture at each of the hip, knee and ankle.

FIG. 1D Is a side elevational view of the implementation shown in FIG. 1C.

FIG. 2 is a side elevational view of the schematic system of FIG. 1A showing about 45° flexion at the knee.

FIG. 3 illustrates the degrees of freedom within a femoral strut or a tibial strut.

FIG. 4 is a block diagram of electronic circuitry.

FIG. 5 is a schematic view of a rotary encoder that may be utilized to determine angulation at a joint.

FIG. 6 is a block diagram of sensor electronics which may be carried within or attached to a joint module at the hip knee or ankle.

FIG. 7 is a block diagram of a remote display unit.

FIG. 8 is a block diagram of a bilateral biometric measurement system.

FIG. 9 illustrates an electronic computing environment according to an embodiment of the present invention.

FIG. 10 illustrates a block diagram of a parameter processing system including example inputs and outputs.

FIG. 11 is a schematic overview of one system in accordance with the present invention.

FIG. 12 is another schematic overview of a system in accordance with the present invention.

### DETAILED DESCRIPTION

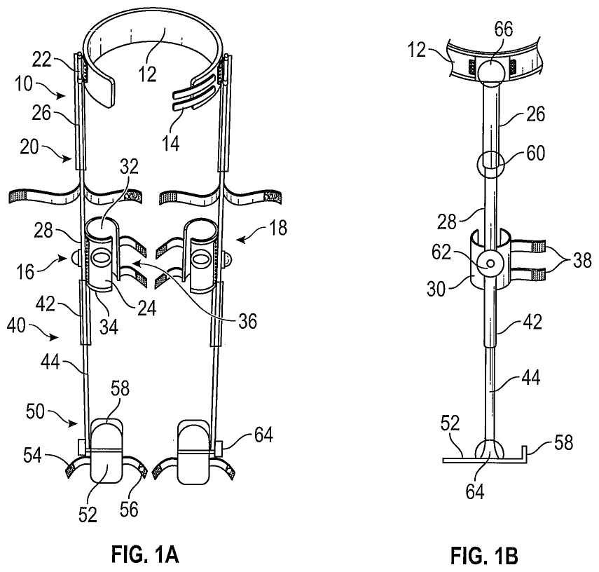

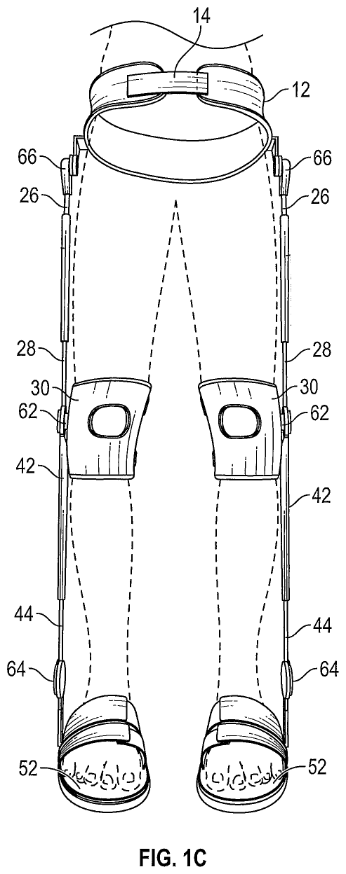

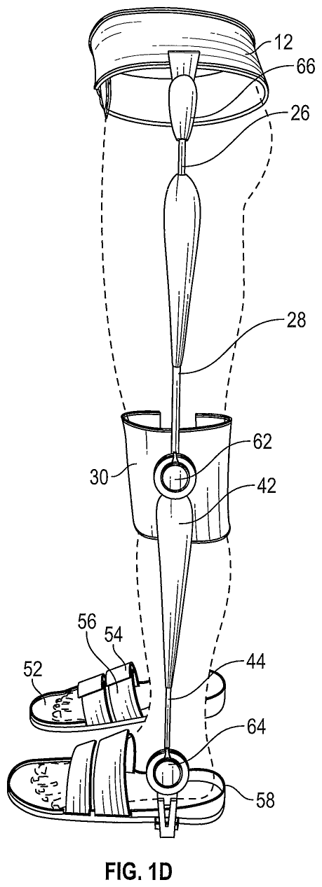

Referring to FIG. 1, there is disclosed in accordance with one aspect of the present invention a physical therapy monitoring system 10. An antimicrobial waistband 12 is provided with at least one fastener 14 such as a hook and loop or other connector which enables rapid attachment and detachment, as well as accommodation of different sized patients. The waistband 12 is connected to a right leg assembly 16 and a left leg assembly 18. The patient monitoring system 10 may be unilateral left or right side depending upon the clinical need. Alternatively the system may be bilaterally symmetrical to capture both left and right side data, in which case the left side components may be the same as the right side components and will not necessarily be separately described herein.

The system may be configured for rapid attachment to the outside of a pair of pants or other athletic gear, or beneath clothing such as street clothing, or may represent a template for a subassembly to be integrated into a garment, and may be provided with any of the resistance elements and/or biometric features (e.g., range of motion, tibial rotation, force, femoral rotation or other stride biomechanics measurement) disclosed elsewhere herein.

Right leg assembly 16 includes a femoral strut 20 which extends between an attachment point 22 at the waist band 12 and a right knee attachment 24. Right femoral strut 20 preferably includes a first component 26 and a second component 28 which are axially movably adjustable with respect to each other. In one specific implementation, at least one of first component 26 and second component 28 are tubular bodies axially movably adjustable with respect to each other such as by telescopic extension and retraction. This enables adjustment of the distance between the waistband 12 and the right knee attachment 24 to accommodate different femur lengths. In the illustrated implementation, the entire femoral strut is tubular, to accommodate electrical wiring as will be discussed.

The right knee attachment 24 may be any of a variety of cuffs or straps such as a split flexible cuff 30 configured for attachment by surrounding the knee across the joint. In the illustrated implementation, cuff 30 is provided with a first flap 32 and a second flap 34 separated by an opening 36 to accommodate rapid positioning and attachment over the knee. At least one connector such as a strap 38 may be utilized to connect the first flap 32 and second flap 34 to enclose the knee and secure the patient monitoring system 10 to the patient.

A right tibial strut 40 is provided, preferably having a first tibial segment 42 and a second tibial segment 44 axially movable adjustable with respect to each other to accommodate different tibial lengths. In one implementation, the first and second tibial segments are coaxially oriented telescopic tubes.

A right foot plate 50 is configured for attachment to a patient's right foot. In one implementation, the foot plate 50 comprises an upwardly facing support surface 52 configured to fit beneath the foot or beneath a shoe worn by the foot. At least one attachment feature such as a first strap 54 and optionally a second complementary strap 56 may be wrapped around the top of the foot and secured to attach the foot to the foot plate. An upwardly extending heel stop 58 may be provided at the posterior of the foot plate 50, to facilitate proper positioning of the foot on the foot plate.

Sensors may be provided at least at one or more of a hip module 60, a knee module 62, and an ankle module 64. Additional sensors and/or electrical components may be carried by a waist band module 66. The sensors may be configured to determine, among other things, anterior-posterior motion; medial-lateral motion and rotational motion such as relative rotation of the second femoral segment 28 with respect to the hip or the first femoral segment 26, or the second tibial segment 44 with respect to the first tibial segment 42, and the foot plate 50 relative to either the right tibial strut 40 or the right femoral strut 20. Thus, the system 10 can determine any of a variety of metrics such as lower extremity range of motion, femoral rotation, tibial rotation, foot rotation, flexion, extension, supination and pronation, step count, distance traveled, power output, among other metrics.

The hip module 60 may be provided with at least one sensor for determining flexion, extension, abduction, adduction and rotation at the hip. The knee module 62 may be provided with at least one sensor for determining similar metrics as at the hip. The ankle module may be provided with at least one sensor for determining polyaxial bending angles, to separately capture all of the natural degrees of freedom of the ankle, such as plantarflexion, dorsiflexion, pronation and rotation.

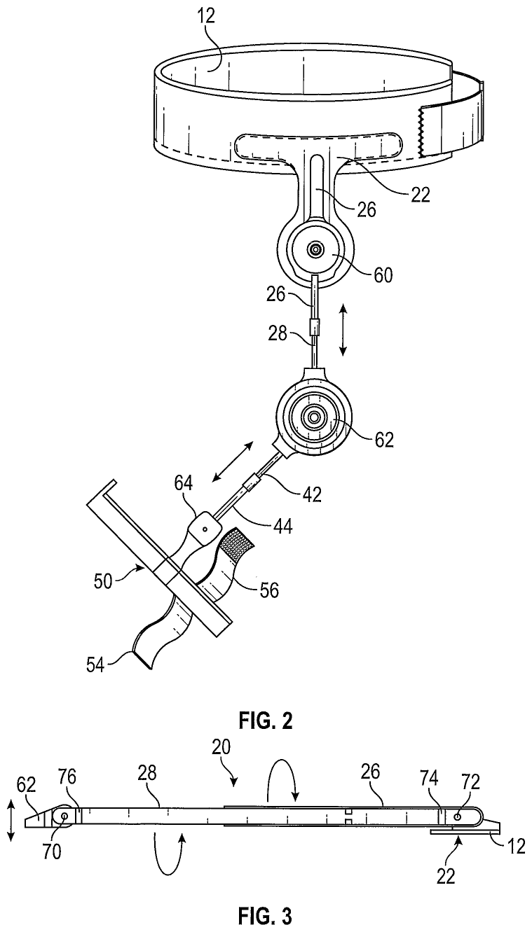

Referring to FIG. 2, there is illustrated a right side view of the monitoring system in accordance with the present invention, in about 45° of flexion at the knee.

The system 10 may be provided with on-board electronics configured solely as a data capture biomechanics unit, to be downloaded following the PT exercise period. It may alternatively be configured as both a data capture, display and/or transmit device, such as to display and/or transmit raw or processed data to a remote receiver, with or without any direct feedback to the wearer. The remote receiver may be a smart phone, tablet or desktop computer, wrist watch or other device capable of receiving and displaying the data, for use by the wearer, a coach, clinician, medical personnel, or anyone who has access to see performance metrics.

FIG. 3 is a schematic illustration of the femoral strut, indicating the degrees of freedom configured to track femoral motion. A first component 26 may be in the form of a tubular sleeve, for axially movably and rotationally receiving a second component 28. An inferior end of second component 28 may be connected via a pivot 70 to the knee module 62. The superior end of the femoral strut is connected via a pivot 72 to the point of attachment 22 at the waist band 12.

At least one sensor 74 may be provided to measure rotation about the pivot 72, reflecting lateral or medial angulation at the hip. At least one sensor 74 may additionally be configured to determine relative rotation between the attachment point 22 and the knee module 62, such as between the first component 26 and second component 28 of the femoral strut 20. At least one sensor 76 may be provided such as at the inferior end of the femoral strut 20, to determine rotation and/or angulation between the femoral strut 20 and the knee module 62. Suitable sensors include rotary encoders, Hall effect sensors and others disclosed elsewhere herein, which may be implemented in pairs having perpendicular axes of rotation. At least one sensor may be provided to measure the flexion and extension angle at the hip

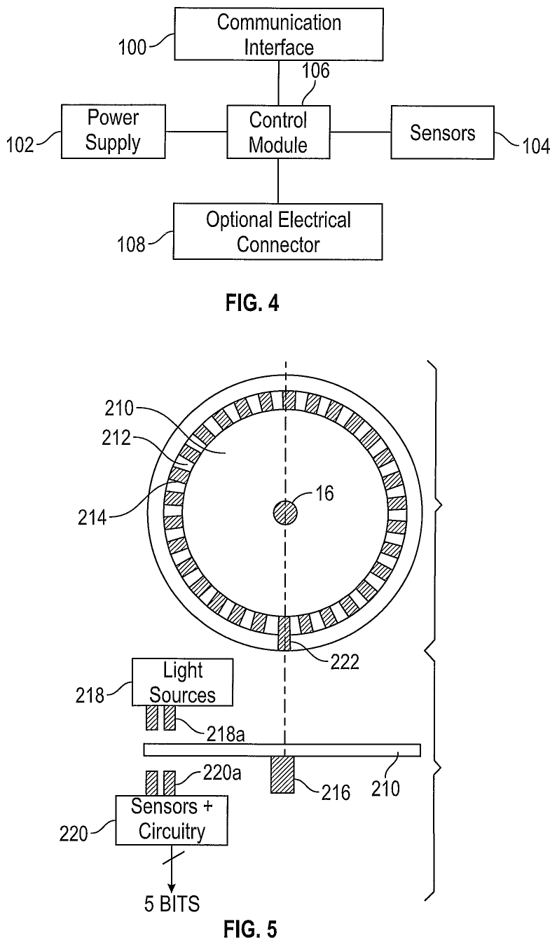

Referring to FIG. 4, a communication interface 100 may be configured to permit electronics on at least one module and/or carried elsewhere on the support (e.g., garment) to communicate (via wired or wireless connection) with one or more of external, remote devices such as a smart personal communication device (e.g., a smart phone, tablet, or pad), remote feedback device, on board feedback device such as a haptic (vibrator), compression pad or ring, electrical current or other feedback effector, or any of a variety of tracker systems such as those produced by Fitbit, Jawbone, Nike's Fuelband or Under Armour's Healthbox connected ecosystem.

Typically, wireless communication among components of the wearable fitness ecosystem may employ any suitable air interface, including for example Bluetooth™ (in its various implementations, including low power Bluetooth), ANT.™, ANT+, WiFi.™, WiMAX.™, 802.11(x), infrared, cellular technology (such as for example GSM.™, CDMA.™, 2G.™, 3G.™, 4G.™, 5G.™, LTE.™, GPRS.™), etc. The selection of the appropriate air interface for communication depends on the air interface availability in the devices and/or at the location, cost, convenience, battery life and/or other factors.

Power supply 102 may comprise a battery pack, which may be carried within the housing of a joint module in a permanent or detachable manner. The battery pack may represent a one-time-use, disposable battery or may represent a rechargeable battery pack (e.g., Lithium-Ion, Nickel Metal Hydride, or the like) to be recharged for use via a charging port (e.g., a micro USB connector) provided with a water resistant cap or plug. Charging may alternatively be accomplished via a wireless charging technology such as inductive charging via an induction coil carried by or within the module housing. The power supplies will be positioned in a manner where there will be no limitation or interference to the movement of the joint.

The battery pack (rechargeable or otherwise) may be configured to be replaceable (e.g., by the user) in the event the battery fails or to swap out a battery with low charge or no charge, with a freshly charged battery, for example. A battery pack support frame carried by a module housing or by the waist band 12 may be configured to accept batteries with different amp-hour capacities to provide sufficient duration of operation of the garment and its associated electronics, such as 1500 mAh, 3000 mAh, etc. Power supply 102 may alternatively comprise an on board generator, such as a rotational energy scarenging generator positioned at the hip or knee to take advantage of reciprocating joint rotation. Other energy scavenging sources can take advantage of body temperature, respiration, stride (e.g., foot strike) temperature change representing calories burned as a result of movement at the hip or others as is understood in the art.

A sensor module 104 can include any of a variety of sensors described elsewhere herein, depending upon the desired functionality. For example, temperature sensors may be provided both to enable correction of other sensor data or electronics (e.g. strain gauges) due to thermal drift as the joint module rises in temperature, as well as to provide biometric data. Sensors for enabling the determination of rotation, pronation, range of motion, force, power, stride length, stride velocity, stride rate, acceleration among others may be conveniently placed on or within the hip module 60, knee module 62 or ankle module 64.

For example, at least one or two or four or more accelerometers, gyroscopes, rotary encoders or other sensors disclosed elsewhere herein may be placed in a module or elsewhere throughout the wearable support (e.g., left and right arm; left and right leg) and/or otherwise carried by the wearer's body (i.e., attached via any suitable manner to shoes, wrist bands, etc.) to collect multiple data points. Each of the additional accelerometers or other sensors may be connected wirelessly or via electrical conductors back to a control module 106 which includes a processor, along with communication module 100 and power supply 102. A suitable 3-axis accelerometer may be a model ADXL377 available from Analog Devices, Inc. of Norwood, Mass. or any equivalent. Likewise, a suitable 3-axis gyroscope may be a model ADXRS652 available from Analog Devices, Inc. of Norwood, Mass. or any equivalent.

Raw data may be sent from an accelerometer and/or a gyroscope and/or a rotary encoder, for example, to the processor which can process and record rotation, acceleration, 3-axis gyroscope position in terms of x, y, and z coordinates which may be used in combination with rotary encoder data. The processor may obtain position point time and angle recordings multiple (e.g., at least about 100 and in one implementation at least about 300 or about 500) times a second and is configured to automatically write the data points to memory along with transmitting the data over the communication interface 100 to sensor data interpretation software which may be resident on a remote computing device (e.g., laptop, cell phone, etc.). Additional details of wearable gyroscope and accelerometer systems may be found in US patent publication 2014/03133049 to Doherty, the entirety of which is hereby expressly incorporated by reference herein.

The control module 106 may also include processing electronics for performing some or all required signal processing on the sensed signals. In one or more embodiments, such signal processing (e.g., amplifying or filtering) may be performed locally in one or more of the sensors, at the control module 106, or both, for example. Control module 106 may also include signal processing capabilities for performing data analysis and feedback data generation. In one or more embodiments, such data analysis and feedback data generation may be performed at one or more of the processor, local remote device such as a fitness tracker or smart phone or the Internet. Signal processing for performing data analysis and feedback data generation may occur solely in the monitoring system 10 (garment) and its associated electronic circuitry, external to garment, or both where some portion of the processing is done in the garment and other portions are done external to the garment using processors and resources of external devices and/or systems.

Control Module 106 may include one or more processors, multi-core processors, one or more digital signal processors (DSP), one or more micro-processors, one or more micro-controllers, one or more application specific integrated circuits (ASIC), one or more field programmable gate arrays (FPGA), one or more analog-to-digital converters (ADC), one or more digital-to-analog converters (DAC), a system on chip (SoC), one or more operational amplifiers, custom logic, programmable logic, analog circuitry, mixed analog and digital circuitry, or the like, just to name a few. Alternatively, raw or partially (incompletely) processed sensor data can be transmitted to an electronics module carried elsewhere, such as on a belt, or off board to a cellphone or other smart local remote device where data manipulation is accomplished. This shifts the weight, power consumption and expense of computational components off board of the garment.

Analysis performed either on board the control module 106 or off board may include, in one or more embodiments, comparing a present parameter such as range of motion with the reference range of motion as is discussed elsewhere herein. Other sensor data such as rotary encoders, bend-angle sensor data or accelerometer sensor data may be used to compare parameters such as acceleration, velocity, other motion or position to the reference data.

Analysis may also include, alternatively or additionally updating a user profile and comparing against profiles of one or more other users. In one embodiment, user profile data may include a history of workout or therapy sessions. In another embodiment, profile data may include goals set by the physical therapist or patient and additionally or alternatively challenges from other users (e.g., to motivate the user). For example, the challenges may come from other persons or users who may be associated with a social network (e.g., Facebook.®, Twitter.®), professional network (e.g., LinkedIn.®), training partner, training team, coach or the like. Through social and/or professional networking of user profiles including historical workout data, motivation is increased by the competitive environment created. This can also be used to assess and monitor compliance with exercise programs that will be performed outside of the clinic such as in a home exercise program or during a telehealth visit.

Additionally, challenges or goals may be proposed by the system 10 (e.g., processor 106 and/or other system in communication with processor 106). A combination of progressive challenges (e.g., a series of challenges, each with higher goals to be achieved) These challenges will all be part of the prescription plan of a licensed therapist or clinician, limiting the chance of injury by overambitious patients or family members may lead the user to higher and higher levels as in a gaming scenario where gamification of the challenges may comprise the user taking on progressive challenges against goals set by the user, the system, others, or by other competitors in the game, for example.

As will be apparent to those of skill in the art in view of the disclosure herein, certain sensors are preferably mounted elsewhere on the garment but other sensors may be or preferably are mounted at or near the axis of joint rotation on the sensor modules. These may include rotary encoders, hall effect sensors, force sensors, angular displacement sensors, accelerometers, proximity sensors, and temperature sensors, such as to calibrate for thermal drift or to directly measure caloric burn accomplished by the resistance unit. All sensors will have the ability to communicate with the control module 106 to process angular motion and force generation during gross functional movements related to activities of daily living (ADL's).

An optional external electrical connector such as a mini USB port may also be provided on the joint module housing, for electrical connection to an external device such as to charge the power supply 102, program the CPU, and or download data which has been obtained during a therapy period or other data collection period. The control module 106 may contain memory, and or a separate memory module may be provided depending upon the intended length of the data collection period and or the complexity (i.e., data rate) of the data being recorded.

One or more sensors 104 carried by one or more of the hip, knee and ankle modules or elsewhere such as adjacent a muscle of interest can include, for example, electromyography (EMG), electrocardiograph (ECG), respiration, galvanic skin response (GSR), temperature, acceleration, bend angle, pressure, force, torque, GPS, accelerometer (single or multi axis), respiration, perspiration, bioimpedence, gyroscopes, various rate measurements such as stride rate, flex rate, pulse (heart) rate, blood pressure, spatial orientation, deviation or position, oxygen saturation, blood glucose, or others described elsewhere herein.

Sensors 104 may also be provided to detect, measure and/or sense data which is representative of hydration, sun exposure, blood pressure and/or arterial stiffness. See, for example, U.S. patent application Ser. No. 14/476,128, filed on Sep. 3, 2014, entitled “Biometric Monitoring Device Having a Body Weight Sensor and Methods of Operating Same” published as U.S. 2014/0379275, which is expressly incorporated herein by reference in its entirety.

The use of multiple sensors 104 for the same parameter or multiple sensors 104 for multiple parameters may provide a level of insight that is not available by measuring only a single metric such as heart rate (HR) or motion based on accelerometers or other types of motion sensors (e.g., a gyroscope). Sensors 104 may be incorporated in a permanent manner into the system itself or in a detachable manner such as with zippers, snap fit connectors, clasps, hook and loop (Velcro) or other releasable connectors and/or in pockets or under or on top of flaps if desired, to allow removal and/or repositioning of the sensors.

A variety of exerted power sensors 104 are known in the performance bicycle arts, which may be readily adapted for use in the present context. Typically, a power sensor such as a strain gauge will be positioned such that it captures force exerted by the wearer. Power sensors may be positioned in a variety of locations on the monitoring system, such as on the lower limit of the system (knee or ankle), and/or carried by any of the sensor modules associated with a joint, like the hip. All may be provided with wired or wireless communication back to a central processing unit carried by the system, or to a remote device such as the activity tracker, cell phone, or other as has been described. Power output by the wearer is perhaps most conveniently measured by utilizing the relative rotation of the femoral strut with respect to the hip and the tibia. Alternatively and/or in addition, wireless power output sensors may be positioned elsewhere in the garment, and configured such as those disclosed in United States patent publication 2015/0057128 to Ishii, the disclosure of which is hereby incorporated by reference in its entirety herein.

Force sensors may be configured to measure force at the point of rotation underlying each of the sensor modules. Signals from any or a combination of sensors 104 may be used to calculate a metric of power (e.g. force or proximity) expended by the wearer. One system having strain gauges embedded in the hub of a rotating construct for the purpose of measuring power is disclosed in U.S. Pat. No. 6,418,797 to Ambrosina et al., the disclosure of which is hereby expressly incorporated in its entirety herein by reference. In another construction, a rotatable axel or post within the joint module is configured to undergo slight deformation in response to applied torque, and sensors are positioned to measure strain as that deformation occurs. Additional details may be found in U.S. Pat. No. 6,356,847 to Gerlitzki, the disclosure of which is hereby expressly incorporated in its entirety herein by reference. Force or power data can alternatively be sent to the processing electronics from other sensors such as sensors carried by or mounted within the wearer's shoes.

As an alternative or in addition to sensors that directly measure force, a variety of position sensors can be incorporated into the system 10. The position sensors are configured to detect angular orientation at the hip, knee, ankle or other measured joint or motion segment and time, and its rate of change. Suitable sensors will communicate with each other and/or control module 106 to better assess how all joints work in tandem while the wearer is performing gross movements for activities of daily living. Suitable sensors for any of the modules include a capacitive transducer, a capacitive displacement sensor, an eddy-current sensor, an ultrasonic sensor, a grating sensor, a Hall effect sensor, a magnetic sensor, an inductive non-contact position sensor, a linear variable differential transformer (LVDT), a differential transformer, a linear variable displacement transformer, a linear variable displacement transducer, a multi-axis displacement transducer, a photodiode array, a piezoelectric transducer, a potentiometer, any of a variety of rotary encoders, a string potentiometer, or a small CCD or CMOS video camera, depending upon the desired performance of the system 10.

A variety of angular position and time sensors such as rotary encoders (e.g., capacitive, magnetic, optical, variable resistance, transmissive, reflective, etc.) may be utilized to capture angular position and time. For example, FIG. 5 schematically illustrates an optical incremental encoder that may be incorporated into each of the joint modules of the present invention. The rotary encoder comprises a disk 210 comprising at least one annular ring of alternating clear and opaque sections 212, 214. The disk can be formed from glass, plastic or metal among other materials, and the clear (transmissive) and opaque sections 212, 214 can be formed by etching, printing, embossing or any other suitable method. The disk 210 is fixed to a pin 216 which may be secured with respect to the femoral strut 20, such that the pin and disk rotate in use relative to a housing which is fixed with respect to the wearers' pelvis in the example of a hip module 60. Referring to FIG. 5, the disk 210 is interposed between one or more light sources 218 and one or more corresponding sensors 220 with associated circuitry, and positioned such that the light sources 218 and sensors 220 are arranged on either side of the circumferential edge comprising the clear and opaque portions 212, 214. In a reflective configuration, the light sources and sensors may be positioned on the same side of the disc.

As the disk 210 rotates in response to movement across a joint such as the hip, the intensity of light incident on the sensors varies as the clear and opaque patterns 212, 214 pass under the light sources 218 in sequence. The measured intensity is amplified or fed into a comparator to produce a sign wave or digital square wave. The pulses of this output are then counted by the circuitry associated with the sensors 220 to give positional information as the intensity of the light varies.

In order to yield an absolute angular position, a known reference point is provided. This can be in the form of a single opaque section 222 on an outer track of the disk 210 or by use of a separate mask component as is well known in the art. In one embodiment, a first light source and sensor pair is provided for detecting the varying intensity of the track comprising the alternating clear and opaque parts 212, 214, and a second light source 218a and sensor 220a pair is provided for the detection of the reference point 22. Additional details may be found in U.S. Pat. No. 7,777,879, the entire contents of which are hereby expressly incorporated by reference herein.

In a capacitive rotary encoder, a disc is provided with a pattern of (typically sinusoidal) metal lines and mounted to a drive shaft for rotation between a transmitter and a receiver. As the central disc rotates in response to a stride, rotation, or flexion/extension exercise of the wearer, the capacitance between the transmitter and receiver changes, thus providing time and position information about the angular orientation of the disk relative to the housing.

A suitable resistive bend sensor (goniometer) is disclosed in Kramer, et al, U.S. Pat. Nos. 5,047,952 and 5,280,265, which are hereby incorporated in their entirety herein by reference and may be incorporated into the systems of the present invention. These sensors may be in the form of thin strips for example approximately 0.01″ thick, 0.20″ wide (minor axis), and variable length (major axis). The sensor measures the angle between the tangents at its endpoints when the sensor assembly experiences pure bending about the minor axis and may be utilized to accurately measure 1, 2 or 3 degrees of freedom, as appropriate, at each joint.

The determination of a biometric parameter can be accomplished on only one of the right side or left side of the wearer, such as at the right hip or hip plus knee but not the opposing side. Preferably, the sensor system will be bilaterally symmetrical on both the right and left side of the wearer, to allow the wearer to see separate values for right and left side performance or an indication of deviation between the right and left sides. This enables use of data from the contralateral side to serve as a control for data from the surgical side in for example a total joint replacement, or fracture fixation resulting in an altered weight bearing status post surgery.

Based at least in part on torque and angular velocity of the leg of the wearer, instantaneous, average, peak, maximum, and/or minimum, horizontal and vertical power exerted by the wearer can be determined and displayed or utilized for further data processing operations such as to generate ratios as is discussed elsewhere herein. Total energy or power exerted by the wearer can be approximated based at least in part on one or more of the wearer's weight, BMI, stride rate, stride length, height, running speed, or any combination of these. These values can be provided to the wearer or care provider to provide feedback regarding power exertion during exercise.

Resistive torque (e.g., a resistance to movement at the hip, knee and/or ankle) may also be provided by resistance units in the joint module. In rotary viscous damper type resistance units, resistance level is related to the angular velocity and/or angular acceleration at the joint. One or more sensors can be provided to measure the angular velocity. These measurements can be used to determine the resistive torque applied by the resistance unit (e.g., the torque that the wearer needs to overcome to move their thigh). Force needed to overcome resistance of gravity can be adjusted depending if the wearer is in the standing, prone, or supine positions) For example, the resistance unit can have a look-up table or other function that maps angular velocity to resistance or resistive torque. Additional details of resistance technology useful for the present joint modules is disclosed in U.S. Patent Publication No. 2017/0274249 to Moebius et al. which is hereby expressly incorporated by reference in its entirety herein.

In some embodiments, the net torque can be used to determine, measure, or estimate the energy or power exerted by the wearer. The instantaneous power can be determined as the product of the net torque and the instantaneous angular velocity of the wearer's thigh (e.g., P=.tau.*.omega., where .tau. is the net torque and .omega. is the instantaneous angular velocity of the thigh). The peak or maximum power can be determined by sampling the instantaneous power over time (e.g., over at least about 1, 2, 5, 10, 20, 50, etc., strides) and determining a maximum power over that time. Similarly, the peak or maximum power can be determined by sampling the instantaneous power over a number of strides, determining a maximum power within each stride, and determining an average or median of the maximum power over the number of strides. The average (median) power can be determined by averaging (determining the median of) measurements of the instantaneous power. Similar processes can be employed to determine other statistical properties of the power. Furthermore, similar calculations and procedures can be followed for determinations of energy or mechanical work exerted by the wearer.

If the angular velocity is not measured or otherwise determined, the instantaneous angular velocity can be estimated in a variety of ways. Some methods for determining instantaneous angular velocity include determining a stride rate and then calculating an estimated instantaneous angular velocity based at least in part on statistical models associating stride rate with thigh position. In certain implementations, the stride rate can be estimated based on a plurality of measurements of torque. The measurements of the torque can be used to estimate the stride rate of the wearer by identifying cyclical patterns within the torque measurements to determine the beginning and endings of strides of the wearer. In various implementations, sensors can be used to determine the stride rate of the wearer (e.g., sensors such as accelerometers, gyroscopes, pressure sensors, or the like can be used). In some implementations, the stride rate can be entered or provided by another system or by the wearer like contact point to determine heel strike and push off within the normal gait pattern

As an alternative to direct measurement, the stride rate can be estimated based on predicted or typical stride rates of runners. For example, a typical recreational runner may have a stride rate between about 150 and about 170 steps per minute. As another example, competitive runners typically have a stride rate between about 180 and about 200 steps per minute. As another example, sprinters can have a stride rate that exceeds about 200 steps per minute. The typical stride rate for a person walking can range between about 100 steps per minute to about 150 steps per minute. We should include where we pulled this data from With the stride rate determined or estimated, the instantaneous angular velocity can be determined based at least in part on a statistical model of the relationship between a phase of the stride and thigh position. For example, the thigh position at various relative times within a stride is statistically similar across adults. Note: thigh angle as described below describes the motion of hip flexion. This can depend at least in part on the speed of the wearer's gait (e.g., walking, running, sprinting, etc.). A walking adult typically has a thigh angle that varies about 50 degrees (e.g., between about 45 and about 55 degrees, or between about 40 degrees and about 60 degrees) over a single stride. A running or jogging adult typically has a thigh angle that various about 55 degrees (e.g., between about 50 and about 60 degrees, or between about 45 degrees and about 65 degrees) over a single stride. A sprinting adult typically has a thigh angle that various about 60 degrees (e.g., between about 55 and about 65 degrees, or between about 50 degrees and about 70 degrees) over a single stride. A competitive sprinter may have a thigh angle that various about 80 degrees (e.g., between about 75 and about 85 degrees, or between about 70 degrees and about 90 degrees) over a single stride. The thigh position as a function of percentage of a stride is typically similar for similar speeds as well. Based on the function of the thigh position as a function of stride, the angular velocity can be estimated (e.g., as a derivative or an approximation of the derivate of the function of the thigh position). These angles will all be considered and calibrated for based on the wearers rehabilitation diagnosis. I.E. normalized to their non effected or surgical side.

The typical total mechanical energy exerted by a person while walking or running can be determined based on the speed, the weight, and/or the stride rate of the person. In various implementations, the mechanical energy exerted by a person can be calculated based on a speed of the person using a statistical relationship. An example statistical relationship of the work done by a person's body, W (in Joules), moving at a speed, x (in meters per second), can be: W=440+170(x−3.3). The variation on this relationship can be between about 10% to about 15% (e.g., the actual mechanical energy has a 68% likelihood of being within 15% of the calculated value using the above relationship). Another example statistical relationship of the work done by a person's body normalized to the weight of the person, Wkg (in Joules/kg), can be: Wkg=7.5+3(x−3.3). The variation on this relationship can be between about 8% to about 12% (e.g., the actual mechanical energy has a 68% likelihood of being within 12% of the calculated value using the above relationship). Another example statistical relationship of the work done by a person's body normalized to the weight of the person and to their stride rate, Wtime (in Joules/kg/s), can be: Wtime=10.5+5.5(x−3.3). The variation on this relationship can be between about 7% to about 10% (e.g., the actual mechanical energy has a 68% likelihood of being within 10% of the calculated value using the above relationship).

In some embodiments, the mechanical energy can be used to determine estimated total power exerted while walking, running, negotiating steps, squatting, lifting or other therapy exercise. This value can be used as a baseline energy or power and the measurements provided by the resistance units can be used as an addition to this calculated energy or power to provide to the wearer an estimate of the energy or power exerted while walking, running, and/or sprinting. In certain embodiments, the measurements provided by the resistance units can be provided as a percentage of the total mechanical energy exerted by the wearer.

In general, a wide variety of information can be calculated on board and relayed to the wearer, to the wearer and a PT or coach, or to the PT or coach alone for display. Alternatively raw data or partially processed data may be exported to a wearer's remote device, and computations performed thereon. In either event, information such as range of motion, rotation, pronation, actual step count, actual distance traveled for walking, near actual distance traveled for running, actual stride length, actual stride rate and real time ratios discussed below can be displayed to the wearer, in many instances more accurately than conventional activity trackers which must in many cases estimate metrics with more or less accuracy.

Certain ratios or relationships can be determined and displayed in real time, and/or saved for later study. For example, power to weight ratio, expressed as watts per kilogram can really be derived and displayed. The controller may be configured to display the trend line over a time interval such as one week, one month, over the season or longer. An athlete can observe an improvement resulting from either a weight loss, an increase in power output, or probably most likely some of both.

Power to heart rate ratio may also be derived and displayed, and utilized for example to determine aerobic decoupling. Aerobic endurance is an important factor in achieving success whether an endurance athlete or a rehabilitation patient Thus, it can be an important training tool to understand whether you have reached an optimal aerobic fitness level. When aerobic endurance improves, there is a reduced upward heart rate drift relative to a constant power output. The reverse is also true that when heart rate is held steady during extensive endurance training, output may be expected to drift downward. This relationship between heart rate and power output is referred to as coupling or power working capacity (PWC).

The extent of decoupling can be quantitatively evaluated during workout in two different ways. If an endurance event is undertaken in such a manner that maintains a steady heart rate, the rate of downward power drift can be monitored. Alternatively, since incremental power (power drift) can be determined essentially in real time in accordance with the present invention, an athlete can focus on maintaining a steady power output and view what happens to heart rate over the measurement period.

Excessive decoupling (too steep a heart rate climb at constant power output or too steep a power decline at constant heart rate) would indicate a lack of aerobic endurance fitness. The controller may be configured to generate comparative displays of the most recent efficiency test with the same test on a prior occasion. The prior occasion may be at least one day, one week, one month, one season or one year or more (e.g., lifetime to date) previously. This information can be utilized to reinforce the value of or modify any of a variety of variables ranging from different types and intensities of training to diet, body weight among others.

An athlete or rehabilitation patient can also utilize the present invention to determine an ideal (e.g., walking, running or cycling) pace. If an athlete or rehabilitation patient is exerting a constant power output, but heart rate is climbing, that exertion level may be acceptable for a short burst but is not sustainable over the long term. Thus the athlete should back down to a lower exertion level. Alternatively, if at a constant power heart rate is declining, the athlete or rehabilitation patient knows that they have a reserve and can afford the energy expense of elevating their exertion level.

Another derived metric that can be determined by the control module 106 for display is efficiency factor. Efficiency factor is normalized power divided by average heart rate over a set interval. By comparing efficiency factor data points over time, such as comparing a present value to a value determined at least one week ago, one month ago, from the beginning of the season, at least a year ago or other interval, one would hope to see an improvement in efficiency factor and can also observe the rate of improvement over time. One will see an improvement in efficiency factor either by experiencing a lower average heart rate for a given steady power output, or an increased power output for a given steady heart rate.

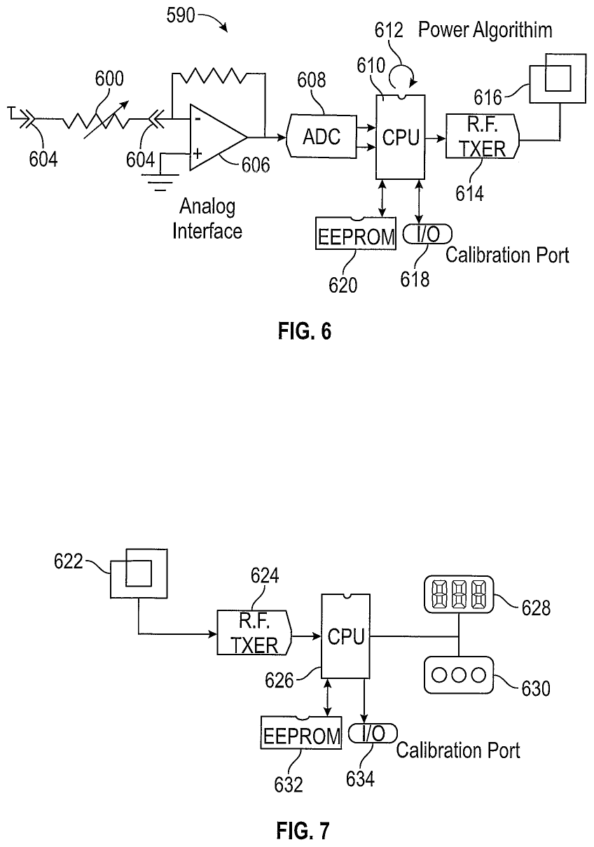

A block diagram showing functional components of an electronics unit 590 is shown in FIG. 6. Force sensor 600 is connected via wire or wireless interface 604. A sensor such as a Flexiforce sensor (obtained from Tekscan of South Boston, Mass., www.tekscan.com) may be used, having a conductance which is linear with force, and an analog interface 606 is used to generate an output voltage that is linear with the applied force. Other analog interfaces may not generate an output voltage that is linear with force, but they will generate a voltage that has a predetermined relationship to a force sensed by the force sensor. The analog interface 606 may contain a variable reference circuit for adjusting a range of the output voltage, depending on the desired performance.

The voltage output by the analog interface 606 drives an analog-to-digital converter 608, which is controlled by a central processing unit (CPU) 610 and sampled at a known and constant rate. The CPU 610 may be, for example, a microprocessor or a digital signal processor. The CPU 610 is responsible for executing a power algorithm 612 that calculates the wearer's power exerted to overcome the resistance element based on force sensed by the force sensor 600.

Data resulting from the calculation is transmitted to a remote electronics unit (activity tracker, cell phone, heads up display, wrist worn display, internet, etc.) by a radio frequency transmitter 614 and antenna 616 via a data channel. During calibration mode, calibration port 618 is used to interface to electronics unit 590. EEPROM memory 620 stores data generated during calibration. Operating power is supplied, for example, by a battery driven power supply, which is not shown but is very well known in the art. Some sensors are preferably calibrated (zeroed) and may be susceptible to drift with changing temperature. A temperature compensation circuit (not shown) is preferably included, to determine the temperature of the sensor and compensate for thermally induced error.

FIG. 7 is a block diagram showing functional components of a remote electronics unit that may display data to the wearer, coach, PT or other application. An antenna 622 and a radio frequency receiver 624 receive data transmitted via the data channel. A CPU 626 controls the user interface, which may include a display 628 and potentially controls such as switches 630. Calibration data and user data are stored in EEPROM memory 632. During calibration mode, calibration port 634 is used to interface to the electronics unit. Operating power for the electronics unit may be supplied, for example, by a battery driven power supply, which is not shown but is very well known in the art. Additional details may be found in U.S. Pat. No. 7,599,806 to Hauschildt, the disclosure of which is hereby incorporated in its entirety herein by reference.

Power may be displayed as real time data, peak, average, rolling average or integrated over a predetermined interval of time (e.g., 10 second, 30 second, I minute or more). Display may be visual, such as on a smartphone, activity tracker or other hand held, wrist worn or mounted device. Power may alternatively be displayed on a heads up display such as an eyeglass with heads up display, or audibly over an audio output using a text to voice converter or tactiley via vibrator or electrical stimulation. Display may be configured to provide an indication of crossing a preset value such as when power output moves either above or below a preset upper or lower alarm limit to allow the wearer to control their power output to within a preset zone.

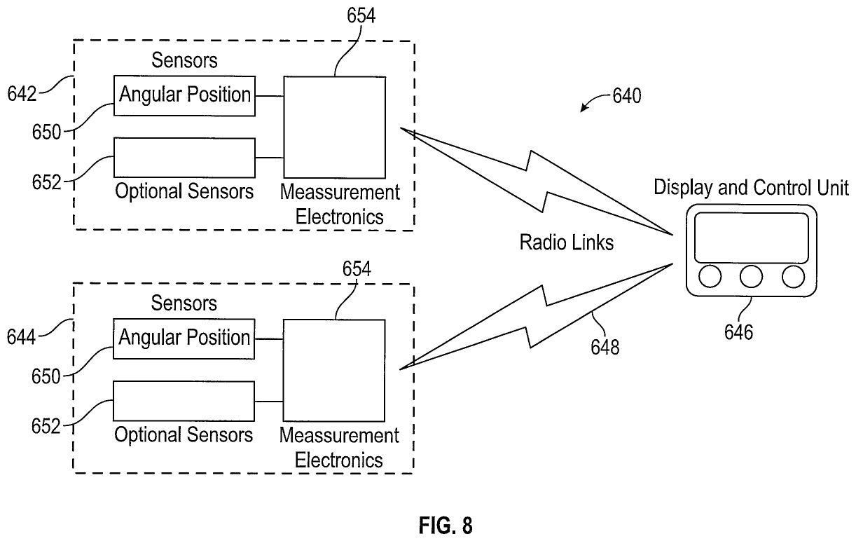

Referring to FIG. 8 there is illustrated a simplified bilateral system to implement the present invention indicated generally by the reference numeral 640. The following discussion of only a left and right pair of sensor modules applies to hip, knee or ankle modules. A left leg sensor module 642 and a right leg sensor module 644 are indicated by dotted lines and are in communication with a control and display unit 646, for example over a radio link 648 (e.g., ANT+, Bluetooth, Zigbee or others disclosed elsewhere herein). Each module 642, 644 comprises of one or more angular position sensor(s) 650, optional additional sensors 652 and related measurement electronics 654 carried by each module. The display and control unit 646, usually battery powered, can be a cell phone or other device carried by the patient or attached to any convenient place such as the wrist, handlebar or other display as has been discussed. The connection between the sensors and electronics in the module and the sensors and electronics elsewhere on or in communication with the garment or wearer may be by wired conductors on or integrated into the garment, or may be by a wireless link such as radio protocols described elsewhere herein or by electromagnetic induction.

In a preferred embodiment the communication between the sensor module electronics and the display and control unit 646 is by a radio link 648. Each of left leg sensor module 642 and right leg sensor module 644 uses the radio to transmit a set of measurement data at one or more fixed points on each stride. In operation each of the sensor modules 642, 644 transmits its data in a short burst when the stride reaches a fixed point in its cycle, such as at the heel strike or toe roll off or end points in a ROM exercise, this can be calibrated to each patient based on diagnosis, BMI, etc

Because the two strides are 180 degrees away from each other in the case of walking or running, data transmission can be timed to ensure that the transmissions from each sensor module assembly will never interfere with each other. Each burst of data contains a set of samples or measurements taken at regular intervals during the stride cycle, and may include ROM, rotation, force, proximity, cadence, femoral (or other) extension angle, heel strike, toe off, and accelerometer information. Each sample has an associated timestamp, which may be explicit or implicit, to specify its time relationship to the other samples in the set and to other sets of samples. The electronics in the sensor modules may include processing of the data before it is transmitted to the control unit 646. Additional details may be found in U.S. Pat. No. 8,762,077 to Redmond, et al., the disclosure of which is hereby incorporated in its entirety herein by reference.

It may be desirable to monitor the wearer's oxygen saturation, and/or CO.sub.2, to evaluate the transition between aerobic and anaerobic threshold as well as the effect on that threshold of varying the degree of resistance provided by the resistance unit (by adjusting an adjustable resistance unit or switching resistance units having different resistance levels). A sensor may be configured to be placed in contact with the wearer such as by permanent or removable attachment to the garment, or independent attachment to the wearer. The sensor may be configured to obtain a plethysmography signal, although it should be understood that any device configured to obtain oxygen saturation and/or heart rate data may be used in accordance with the techniques of the present disclosure.

The system may include a monitor in communication with the sensor. The sensor and the monitor may communicate wirelessly as shown, or may communicate via one or more cables (e.g., the sensor and the monitor may be coupled via one or more cables). The sensor may include a sensor body, which may support one or more optical components, such as one or more emitters configured to emit light at certain wavelengths through a tissue of the subject and/or one or more detectors configured to detect the light after it is transmitted through the tissue of the subject.

The sensor may include one or more emitters and/or one or more detectors. The emitter may be configured to transmit light, and the detector may be configured to detect light transmitted from the emitter into a patient's tissue after the light has passed through the blood perfused tissue. The detector may generate a photoelectrical signal correlative to the amount of light detected. The emitter may be a light emitting diode, a superluminescent light emitting diode, a laser diode or a vertical cavity surface emitting laser (VCSEL).

Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent and the related light absorption. For example, the light from the emitter may be used to measure blood oxygen saturation, water fractions, hematocrit, or other physiological parameters of the patient.

In certain embodiments, the emitter may emit at least two (e.g., red and infrared) wavelengths of light. The red wavelength may be between about 600 nanometers (nm) and about 700 nm, and the IR wavelength may be between about 800 nm and about 1000 nm. However, any appropriate wavelength (e.g., green, yellow, etc.) and/or any number of wavelengths (e.g., three or more) may be used. It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure. It should be understood that all patients will be screened by a licensed medical professional for any comorbidities or past medical history to where these waves will be a contraindication such as a pacemaker.

The detector may be an array of detector elements that may be capable of detecting light at various intensities and wavelengths. In one embodiment, light enters the detector after passing through the tissue of the wearer. In another embodiment, light emitted from the emitter may be reflected by elements in the wearer's tissue to enter the detector. The detector may convert the received light at a given intensity, which may be directly related to the absorbance and/or reflectance of light in the tissue of the wearer, into an electrical signal. That is, when more light at a certain wavelength is absorbed, less light of that wavelength is typically received from the tissue by the detector, and when more light at a certain wavelength is transmitted, more light of that wavelength is typically received from the tissue by the detector. After converting the received light to an electrical signal, the detector may send the signal to the monitor, where physiological characteristics may be calculated based at least in part on the absorption and/or reflection of light by the tissue of the wearer.

As indicated above, the monitoring system may be configured to monitor the wearer's oxygen saturation and/or heart rate during exercise. The system may also be configured to determine whether the wearer is utilizing an aerobic or an anaerobic pathway based at least in part on the athlete's oxygen saturation and/or heart rate. For example, the monitoring system may compare the athlete's or rehabilitation patient's oxygen saturation and/or heart rate to one or more zones corresponding to various types of exercise (e.g., aerobic exercise and anaerobic exercise) to determine whether the wearer is utilizing the aerobic or the anaerobic pathways. Each of the one or more zones may be defined by a percentage or a range of percentages of oxygen saturation and/or a value or a range of values of heart rate, and each of the one or more zones may have an upper limit and a lower limit for oxygen saturation and/or heart rate. Safeguards will be put in for exercise to halt if Heart rate or saturation levels reach a range that is unsafe—this can be pre calibrated based on prescription and limitations.

For example, a first zone may include an oxygen saturation range and/or a heart rate range corresponding to aerobic exercise, while a second zone may include an oxygen saturation range and/or heart rate range corresponding to anaerobic exercise. A visual, audio and/or tactile display or feedback may be provided to the wearer to indicate status and/or change in status between an aerobic metabolism level of activity and an anaerobic metabolism level of activity. Additional implementation details may be found in US patent publication No. 2015/0031970 to Lain, entitled Systems and Methods for Monitoring Oxygen Saturation During Exercise, the disclosure of which is hereby incorporated by reference in its entirety herein.

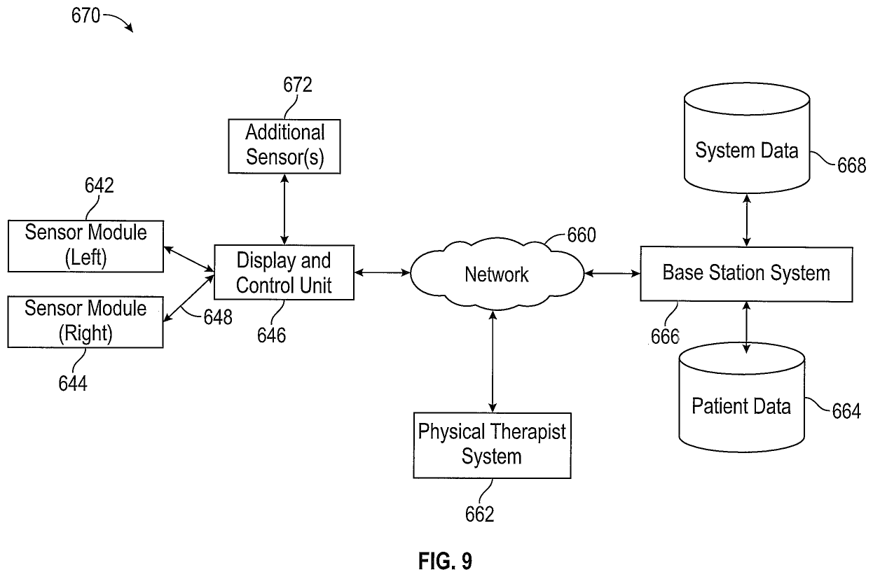

FIG. 9 illustrates an electronic computing environment 670 including interaction and communication between multiple electronic systems. As discussed above, the biometric units such as sensor modules 642 and 646 can transmit data, which is captured by the angular position sensors disclosed herein, to a display control unit 646 over communications link 642 and 648. These communications links 642 and 648 can include radio links for wireless transmission of the captured data to the display and control unit 646. In some embodiments, the display and control unit 646 can include a mobile device including an antenna for receiving the transmitted captured data. In some embodiments, the display and control unit 646 can send instructions to the sensor modules 642 and 644 to transmit the captured data. The display and control unit 646 can also manage bandwidth and power requirements during transmission.

The display and control unit 646 can include a memory to store the received data from the sensor modules 642 and 644. The display and control unit 646 can further include one or more receiving and transmitting antennas and one or more hardware processors. In some embodiments, the display and control unit 646 can also receive data from addition sensor(s) 672. As discussed above, additional sensor(s) 672 can include an oxygen saturation detecting sensor, a heart rate detector, or the like. The display and control unit 646 can transmit the stored data over a network 660 to a base station system 666. The network 660 may be a local area network (LAN), a wide area network (WAN), cellular network, such as the Internet, combinations of the same, or the like. The transmission of data from the display and control unit 646 to the base station system 666 may be automatic or based on a user input.

The base station system 666 can include one or more servers for implementing and executing a parameter processing system 674 as discussed below. The base station system 666 can also include one or more data repositories 664 and 668. These data repositories can store user specific data, including historical data received from the display and control unit 646. The data repositories can also store predefined system parameters including threshold conditions and constants. The electronic computing environment 670 can also include a coach system 662 (PT coaching, athletic coaching) capable of receiving the captured data and output generated by the parameter processing system discussed below. The coach system 662 can include a coaching interface such as a visual display and optional audio user interface and underlying computing devices, including mobile electronic devices as discussed herein.

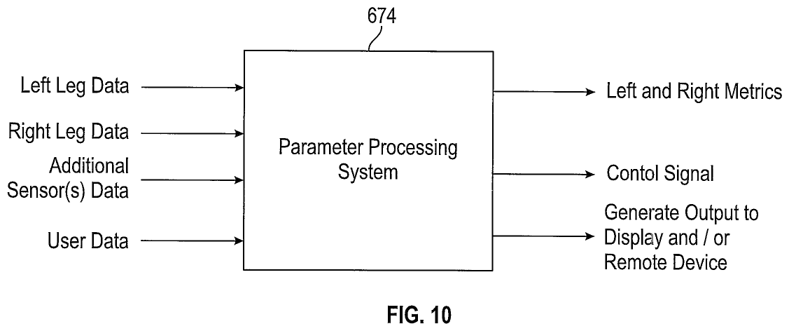

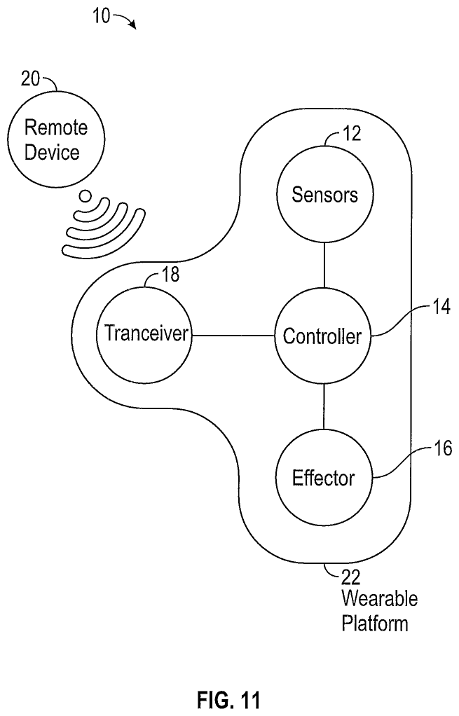

FIG. 10 illustrates an embodiment of a parameter processing system 674 for generating one or more electronic outputs based on received data. Operations can include capturing data, transmission of captured data, and other operations described herein. The parameter processing system 674 includes programming instructions for the control and generation of output. Accordingly, the programming instructions correspond to the processes and functions described herein. The programming instructions can be stored in a memory of the base station system 666. In some embodiments, the programming instructions can also be stored in the display and control unit 646, coach system 662, such that some or all aspects of the parameter processing system 674 can be implemented in the display and control unit 646 and/or the coach system 662. The parameter processing system 674 can be executed by one or more hardware processors in the base station system 666, coach system 662, or display and control unit 646, or a combination. The programming instructions can be implemented in C, C++, JAVA, or any other suitable programming languages. In some embodiments, some or all of the portions of the parameter processing system 674 can be implemented in application specific circuitry such as ASICs and FPGAs.

FIG. 10 illustrates example inputs and outputs generated by the parameter processing system 674. In some embodiments, the parameter processing system 674 can electronically derive total power expended, peak power, power per stride, declining power reserve and other parameters discussed herein. The parameter processing system can also generate displays for display on a coach system 662 or the display and control unit 646. In some embodiments, the parameter processing system 662 generates output in near real time, with minimal delay from the time that the data was captured. In an embodiment, the delay is less than 1 second. The generated display can include power and heart rate ratio data. The parameter processing system 674 can accumulate historic data and determine an average to generate a baseline for the total power expended or other metric experienced per PT session, home therapy session, per play, per game, marathon, or other physical event. The generated display can show a declining remaining power based on the stored historical performance. The display can include display elements, such as a gauge with scales based on the historical performance and the current meter based on captured data.

Any of the metrics identified herein can be captured and expressed as separate left leg and right leg, hip, knee and ankle data, and deviations can be identified and signaled or displayed to reveal bilateral asymmetries which may suggest corrective training and/or progress in the physical therapy or other diagnostic or therapeutic environment. Any or all of the foregoing can be displayed in real time and/or downloaded to a program configured to provide post event or therapy session analytics.

Although disclosed primarily in the context of lower body garments, any of the sensor modules and attachment structures disclosed herein can be adopted for use for any other motion segment on the body, including the shoulder, elbow, wrist, neck, abdomen (core), lower back, pelvis, and various other motion segments of the upper body. Any of the various modules and attachment structures disclosed herein can be interchanged with any other, depending upon the desired performance. In addition, the present invention has been primarily disclosed as coupled to a type of harness or support frame. However any of the resistance systems disclosed herein may be carried by any of a variety of other types of garments including braces, wearable clothing subassemblies, straps, cuffs or other wearable support construct that is sufficient to mechanically couple one or more resistance or data capturing elements to the body and that may be worn over or under or integrated into conventional clothing.

A wearable support such as a form fitting garment or support frame in accordance with the present invention may include at least one sensor and associated processing and communications electronics, and at least one effector which provides feedback to the wearer in response to data collected by the sensor and interpreted by the processor. Additional details of the sensor, feedback and processing can be found in U.S. Patent Publication No. 2021/0084999 to Matsuura et al., the entirety of which is hereby expressly incorporated by reference herein.

Referring to FIG. 11, there is illustrated a schematic representation of a PT monitoring system with a wearer feedback feature in accordance with the present invention. The system 10 includes at least one sensor 12, described in greater detail below. The sensor 12 is in electrical communication with a controller 14. Controller 14 is in communication with at least one effector 16, for providing feedback and/or adjusting a physical parameter of the system such as resistance to movement, compression or other parameter that will be perceptible to the wearer. The controller 14 may additionally be in communication with a transceiver 18, for communication with a remote device 20 which may be a real time or delayed connection with a remote therapist. In certain embodiments, all of the foregoing components are carried by a wearable platform such as a garment 22 or support frame as has been discussed.

Visual, audio or tactile feedback can be provided to the wearer to signal to the wearer that they should adjust their posture, or adjust a performance parameter such as increase or decrease stride length, range of motion or repetition rate, realignment of stride, modify arm swing such as bring the elbows in or other streamlining adjustment, or adjust their spine (core) such as to bring it into alignment with a preset data set, or initiate other motion or body position correction.

In certain implementations of the invention, feedback may be provided to the wearer at preselected end points of motion such as end points of a desired range of motion. For example, feedback such as tactile feedback may be provided upon achieving a flexion of at least about X degrees or an extension of at least about Y degrees. That lets the patient know that they have achieved a desired range of motion end point. That data may be stored for comparing to corresponding data in a subsequent therapy session, in which progressive goals may be set. For example, the feedback may be provided when the patient achieves an extension of X plus A degrees or a flexion of Y plus B degrees. Progressive goals may be set by the processor, or maybe set by the physical therapist, and enable quantitative tracking of rate of improvement.

Any of a variety of effectors 16 may be provided in accordance with the present invention, to provide behavior recording or behavior modifying feedback or to modify garment performance. The effectors may provide visible, tactile, and/or audio feedback, depending upon the desired performance result. Effectors 16 thus may include structures such as vibrators, pressure generators such as inflatable balloons or electromechanical structures such as a solenoid involving motion or application of localized pressure to the wearer. Effectors may also apply an electrical current to the dermal surface, such as to produce an electrical shock or muscular stimulation. Effectors may produce sound, or provide a visual indicator such as a light or information on a local display such as a watch, cell phone, head worn display or other local display device as well as a remote display for the therapist.

Vibrators or other effectors can be carried by the support and be positioned adjacent specific locations on the body which correlates with the nature of the desired proprioceptic feedback. Thus the wearer can receive a perceptible feedback which provides different instructions to the wearer depending upon the location of the effector. Activation may be pulsed or continuous until sensors determine that the desired correction or endpoint has been accomplished. The frequency of pulsed feedback and/or the intensity of the feedback (e.g., vibration) can increase in proportion with the degree to which a target value is exceeded. A stepped feedback protocol can also be programmed, such that a first effector is activated when a first value is reached, and a second effector is activated when a second value is reached, typically at a greater deviation from the desired target than the first value. The value can be any measured parameter of any of the sensors disclosed herein.

A vibrator may be positioned, for example, on any or all of the posterior side of one or both lower or upper legs, activation of which tells the wearer to make a modification such as increase angulation, stride length or tempo. In general, sensors and/or effectors can be placed at any one or combination of the anterior right or left side or the posterior right or left side of the lower leg, upper leg, waist, glut, lower arm or upper arm. On the core or torso, effectors can be located on anterior, posterior or lateral sides, at the cervical, thoracic lumbar or sacral level of the spine, as well as the head, left or right hand or left or right foot. The wearer can be taught to move the portion of the body in the vicinity of a given effector towards or away from the direction of the effector in response to activation, or to accomplish some other behavioral modification in response to each effector activation.

One preferred effector comprises a device which when activated produces tactile sensation such as from a pressure or vibration against the skin. Small effective vibrators can comprise a small motor having a rotatable shaft, with a weight eccentrically carried by the shaft. The weight throws the shaft out of balance and produces vibration of the entire assembly. The vibrators may be permanently mounted on the support, or may be detachable such as for washing, repositioning or replacement. The vibrator and any associated wiring is preferably water proof and can sustain wash and dry cycles. One suitable overmolded, waterproof vibrator is disclosed in US patent publication No. 2014/0265677 to Grand, the disclosure of which is hereby incorporated by reference in its entirety herein.

Effectors may additionally include devices that produce a constriction or expansion of the garment along predetermined planes, in response to measured parameters, For example, at least a portion of a support may be provided with an inflatable reservoir such as a balloon that can be inflated by a small pump, to increase compression against the wearer. Compression can alternatively be achieved using a device or material that can shorten in length upon activation such as NiTinol wires or fabric which shorten upon application of a current, or a motor driven take up spool. Such devices can be used to selectively constrict either around a circumference of a body part such as a waist, thigh, calf, arm, core (abdomen) etc., or along a preselected axis such as to apply compression or to change posture across a shoulder or body core.

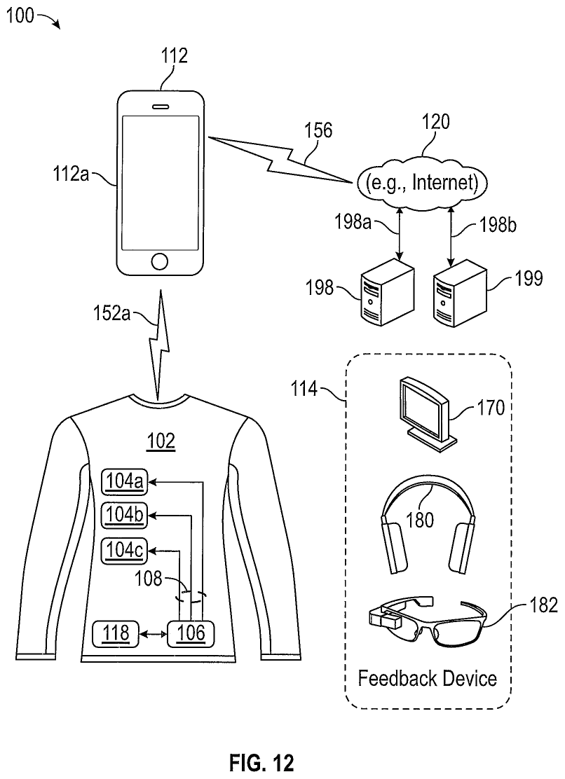

With reference to FIG. 12, there is depicted a form-fitting sensor garment 102, representing a compressive, stretchable, and form-fitting garment to be worn by a human subject (not shown). Although garment 102 is shown to be a shirt, it can take any other garment form factor including but not limited to shorts, yoga pants, compression pants, elbow pad, knee pad, undergarment, neck wrap, glove, and the like, as well as the support frame discussed in detail elsewhere herein. Additional details of the system of FIG. 12 can be found in US patent publication No. 2021/0084999 to Matsuura, et al., entitled Dynamic Proprioception, the contents of which are hereby incorporated in their entirety herein by reference.

Systems of the present invention may be utilized in any of a variety of athletic applications as well as strength and conditioning as example applications. Other applications such as medical physical or neurological rehabilitation or realignment or training of balance or perceived spatial orientation or training a wearer to follow a predetermined routine such as a physical therapy protocol are also contemplated. The end use application in which the sensors and system described herein may be used does not materially change the form or function of the concepts described in the present application.

For example, the ability to critique form, posture, ROM, pronation, etc and provide proprioceptic feedback as discussed herein can be used to train proper procedures in a manufacturing environment, work environment (e.g., functional capacity evaluation, work conditioning, work hardening) and other physical therapy and athletic endeavors, just to name a few. Critiquing posture or movement patterns may provide injury prevention in the workplace in the same way as in an athletic training setting. As another example, in that the present application builds on clinical methods and provides a more user-friendly experience, the present system may be applied for use in self-guided outpatient (telemedicine) medical rehabilitation and injury prevention training.

1. A wearable physical therapy system, comprising:
a wearable support comprising a waistband and at least one motion detector, the motion detector assembly comprising:
a femoral strut extending between a superior end and an inferior end;
a polyaxial connection between the superior end and the waistband;
hip sensors in motion sensing communication with the polyaxial connection, configured to capture data reflecting movement of the femur with respect to the hip in the medial—lateral direction, anterior—posterior direction and rotation of the femur.

2. A wearable physical therapy system as in claim 1, comprising a left leg motion detector assembly and a right leg motion detector assembly.

3. A wearable physical therapy system as in claim 1, further comprising a knee attachment configured to secure the inferior end of the femoral strut with respect to the knee.

4. A wearable physical therapy system as in claim 3, wherein the knee attachment comprises a flexible cuff.

5. A wearable physical therapy system as in claim 1, further comprising a tibial strut extending between the knee attachment and an inferior attachment configured to secure the tibial strut with respect to the ankle.

6. A wearable physical therapy system as in claim 5, further comprising a foot attachment, for securing the tibial strut with respect to a wearer's foot.

7. A wearable physical therapy system as in claim 6, further comprising at least one sensor for measuring pronation of the foot.

8. A wearable physical therapy system as in claim 6, further comprising at least one sensor for measuring flexion or extension of the foot.

9. A wearable physical therapy system as in claim 6, further comprising at least one sensor for measuring rotation of the foot.

10. A wearable physical therapy system as in claim 3, wherein the length of the femoral strut between the waistband and the knee attachment is adjustable.

11. A wearable physical therapy system as in claim 10, wherein the femoral strut comprises first and second telescopically extendable components.

12. A wearable physical therapy system as in claim 11, wherein the first and second components are tubular.

13. A wearable physical therapy system as in claim 12, further comprising a knee module carried by the knee attachment.

14. A wearable physical therapy system as in claim 13, comprising at least one sensor in the knee module, configured to measure flexion and extension at the knee.

15. A wearable physical therapy system as in claim 14, further comprising electrical conductors extending through the femoral strut

16. A wearable physical therapy system as in claim 1, wherein the sensor collects angular position and time data.

17. A wearable physical therapy system as in claim 16, wherein the sensor comprises a rotary encoder.

18.-19. (canceled)

20. A wearable ankle range of motion measuring device, comprising:
a foot support, configured for attachment to a foot;
a tibial support, configured for attachment to a leg;
a polyaxial connection between the tibial support and the foot support, having a first sensor for measuring adduction and abduction, a second sensor for measuring dorsiflexion and planter flexion, and a third sensor for measuring inversion and eversion.

21.-26. (canceled)

27. A computer-implemented method of displaying physical therapy progress comprising, via at least one processor of at least one computing platform operably coupled to at least one storage device and a display device:
providing the at least one storage device containing a first set of user body movements of a user performing a movement routine previously captured via at least one motion tracking apparatus;
capturing a second set of user body movements of the user performing the movement routine; and
presenting data via the display device, the data configured to display a difference between the first and second sets of user body movements to reveal physical therapy progress

28-44. (canceled)
